(19) United States
(12) Patent Application Publication
(10) Pub. No.: US 2019/0057775 A1
(43) Pub. Date: Feb. 21, 2019

(54) System and method of managing patient data of one or More Patients

(71) Applicant: Payoda Technology Inc., Plano, TX (US)

(72) Inventor: Anand Purusothaman, Jersey, NJ (US)

(21) Appl. No.: 15/998,691

(22) Filed: Aug. 16, 2018

Related U.S. Application Data
(60) Provisional application No. 62/546,331, filed on Aug. 16, 2017.

(51) Int. Cl. G16H 50/20; G16H 10/60; G16H 80/00; G16H 20/00; H04L 29/06

(52) U.S. Cl. CPC G16H 50/20; G16H 10/60; H04L 63/10; G16H 20/00; G16H 80/00

(57) ABSTRACT

A system and method for managing data of one or more patients is provided. The method includes the steps of: (a) processing a first set of data and a second set of data to consolidate (i) the first set of data and (ii) the second set of data into a single source data; (b) tagging, by a primary physician, the first set of data and/or the second set of data with selected each of one or more secondary physicians; (c) integrating the patient health management system with one or more health monitoring devices to obtain the data and to communicate the first set of data and/or the second set of data to their the one or more of physicians; and (d) consolidating the first set of data and/or the second set of data in standard formats.

### CLAIM OF PRIORITY

The present application claims priority to U.S. provisional patent application No. 62/546,331, entitled “System and Method for Managing Patient Data of One or More Patients,” filed on Aug. 16, 2017, invented by Anand Purusothaman, the disclosure of which is hereby incorporated by reference for all purposes as if fully set forth herein.

### TECHNICAL FIELD

The embodiments herein generally relate to a data management platform, and more particularly, to a system and method for patient referral management, patient tagging, consolidation and standardization of data of one or more patients.

### DESCRIPTION OF THE RELATED ART

Generally, patients receive healthcare services from several sources, such as physician practices, hospitals, urgent care centres, pharmacies and specialized medical centres. A patient's medical information is fragmented among various proprietary systems throughout these healthcare sources, making it difficult for one provider to access an information originally documented by another provider. In addition, many consumers generate health data while using a rich variety of health-related websites, apps, tools and technologies. Therefore, it is challenging for physicians to construct a holistic picture of the patient's health nor does the patient have means to manage and maintain their complete health information from these various health sources. The main challenge that the healthcare industry is facing is to create a comprehensive view of a patient's health data, through data collation and consolidation from disparate sources, data standardization and finally making it available to the physician with just the amount of information that is required.

One of the current challenges of the health care industry is to provide an innovative way of interaction between patient health data and providers (e.g. physicians). The disparate data sources, systems with non-standard data formats, incompatible terminologies, new/changing regulations and the inability for the existing health systems to cope with it, closed EHR or EMR systems, etc., make it even more complex to solve the health interoperability challenge. With the increasing number of Physician Networks, Independent Physician Associations and distributed Health Systems that are a result of Hospital Systems buying physician groups, deploying an effective patient referral management system with an ability to track and close the referral loop becomes a key requirement to achieve.

Patients typically seek treatment from a physician for newly arising medical conditions and may return to the same physician for follow-up or be referred to a different physician who may then assume responsibility for treating the patient for that condition. Patients may visit more than one physician for the same complaint or consult different specialists for different complaints. Many patients suffer from several chronic diseases, require multiple medications, and are under the care of multiple physicians, who may practice at different healthcare institutions and may not even be aware of multiple disease associated with a particular patient. In addition, physicians may have an incomplete picture of the patient's health due to factors such as patient forgetfulness or lack of time to discuss the relevant questions during patient appointments. The fragmented and intermittent nature of health care is particularly problematic for the increasing number of patients with chronic diseases.

Accordingly, there remains a need for a system and method for centric conversation among physicians, patient data management and referral management for the physicians.

### SUMMARY

In view of the foregoing, an embodiment herein provides a secure healthcare data management server (SHDMS) for secure communication of healthcare data related to a patient among by a primary physician with one or more secondary physicians. The server includes a memory unit and a processor. The memory unit stores a database and set of modules. The processor executes set of modules. The set of modules includes a patient data selection module, a secondary physician selection module, a physician interacting module, a patient data tagging module, an access limit determining module, a diagnosis and recommendation downloading module and a patient data link accessing module. The patient data selection module selects a first patient information among a list of patients by the primary physician. Each physician acts as a primary physician for the list of patients. The secondary physician selection module enables the primary physician to tag the first patient information with one or more secondary physicians based on the field of expertise of each secondary physician. The physician interacting module enables the primary physician to initiate a communication with the tagged one or more secondary physicians for diagnosis or request help in treatment by sharing electronic medical report (EMR) of the patient. The patient data tagging module determines a field of expertise of the tagged one or one or more secondary physicians. The access limit determining module enables the primary physician to limit access to the EMR of the patient to the tagged one or more secondary physicians by selecting each physician separately that is specifically relevant to the field of expertise of the tagged one or one or more secondary physicians. The diagnosis and recommendation downloading module identifies a disease or ailment of the first patient based on the EMR from the primary physician by at least one tagged secondary physician. The treatment recommendation module recommending a customized treatment process for the first patient based on the identified disease of the first patient to the primary physician by the least one tagged secondary physician.

In an embodiment, the physician interacting module enables the primary physician to view a history of communication in a timeline window associated with the one or more secondary physicians associated with each patient in a predetermined time manner.

In another embodiment, the physicians interacting module verifies the one or more secondary physicians by communicating the patient data as a secured link from the primary physician to the one or more secondary physicians. The physician access module authorizes the secure link by the one or more secondary physicians to access the patient data and enables one or more secondary physicians to access EMR through SHDMS when the secure link is accessed by the one or more secondary physicians.

In yet another embodiment, the access limit determining module creates communication groups from tagged one or more secondary physicians based on their field of expertise and the patient data.

In yet another embodiment, the secondary physician selection module enables the primary physician to select the one or more secondary physicians in at least one communication group.

In yet another embodiment, the patient data link accessing module enables at least one selected secondary physician to view and access the patient data in the at least one communication group even if the one or more secondary physicians are present in the group by sharing a secure link to the selected secondary physician.

In another aspect, the computer implemented method for secure communication of healthcare data related to a patient among by a primary physician with one or more secondary physicians using a secure healthcare data management server (SHDMS). The computer implemented method including following steps: (i) selecting a first patient information among a list of patients by the primary physician. Each physician acts as a primary physician for the list of patients, (ii) enabling the primary physician to tag the first patient information with one or more secondary physicians based on the field of expertise of each secondary physician, (iii) enabling the primary physician to initiate a communication with the tagged one or more secondary physicians for diagnosis or request help in treatment by sharing electronic medical report (EMR) of the patient, (iv) determining a field of expertise of the tagged one or one or more secondary physicians, (v) enabling the primary physician to limit access to the EMR of the patient to the tagged one or more secondary physicians by selecting each physician separately that is specifically relevant to the field of expertise of the tagged one or one or more secondary physicians, (vi) identifying a disease or ailment of the first patient based on the EMR from the primary physician, by at least one tagged secondary physician and (vii) recommending a customized treatment process for the first patient based on the identified disease of the first patient to the primary physician by the least one tagged secondary physician.

In an embodiment, the method further includes the step of enabling the primary physician to view a history of communication in a timeline window associated with the one or more secondary physicians associated with each patient in a predetermined time manner.

In another embodiment, the method further includes the steps of (i) verifying the one or more secondary physicians by communicating the patient data as a secured link from the primary physician to the one or more secondary physicians, (ii) authorizing the secure link by the one or more secondary physicians to access the patient data and (iii) enabling one or more secondary physicians to access EMR through SHDMS when the secure link is accessed by the one or more secondary physicians.

In yet another embodiment, the method further includes the steps of (i) enabling the primary physician to select the one or more secondary physicians in at least one communication group and (ii) enabling at least one selected secondary physician to view and access the patient data in the at least one communication group even if the one or more secondary physicians are present in the group by sharing a secure link to the selected secondary physician.

### BRIEF DESCRIPTION OF THE DRAWINGS

The embodiments herein will be better understood from the following detailed description with reference to the drawings, in which:

FIG. 1 illustrates a system view of a patient data management system for managing data associated with one or more patients according to an embodiment herein;

FIGS. 2A and 2B illustrate an architectural view of the patient data management system of FIG. 1 according to an embodiment herein;

FIG. 3 illustrates an exploded view of a centralized healthcare management system of FIG. 1 according to an embodiment herein;

FIG. 4 illustrates an exploded view of one or more infirmary management system of FIG. 1 according to an embodiment herein;

FIG. 5 illustrates an exploded view of one or more patient healthcare management system of FIG. 1 according to an embodiment herein;

FIG. 6 illustrates a user interface view of a patient selection of the one or more, infirmary management system according to an embodiment herein;

FIG. 7 illustrates a user interface view of a tag physicians of the one or more infirmary management system according to an embodiment herein;

FIG. 8 illustrates a user interface view of a patient data sharing according to an embodiment herein;

FIG. 9 illustrates a user interface view of a selectively dispense data according to an embodiment herein;

FIG. 10 illustrates a user interface view of a patient data transfer confirmation according to an embodiment herein;

FIG. 11 illustrates a user interface view of a patient data link sharing according to an embodiment herein;

FIG. 12 illustrates a user interface view of a request for approval according to an embodiment herein;

FIG. 13 illustrates a user interface view of a patient data sharing according to an embodiment herein;

FIG. 14 illustrates a user interface view of a timeline according to an embodiment herein;

FIG. 15 illustrates a user interface view of an electronic health record/electronic medical record (EHR/EMR) view with one or more action according to an embodiment herein;

FIG. 16 is a flow diagram illustrating a method of managing healthcare data using the centralized healthcare management system of FIG. 1 according to an embodiment herein;

FIGS. 17A and 17B are flow diagrams illustrating a method of managing healthcare data using the one or more infirmary management system of FIG. 1 according to an embodiment herein;

FIG. 18 is a flow diagram illustrating a method of managing healthcare data using the one or more patient healthcare management system of FIG. 1 according to an embodiment herein;

FIG. 19 illustrates an exploded view of a receiver of FIG. 1 according to an embodiment herein; and

FIG. 20 illustrates a schematic diagram of a computer architecture used according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

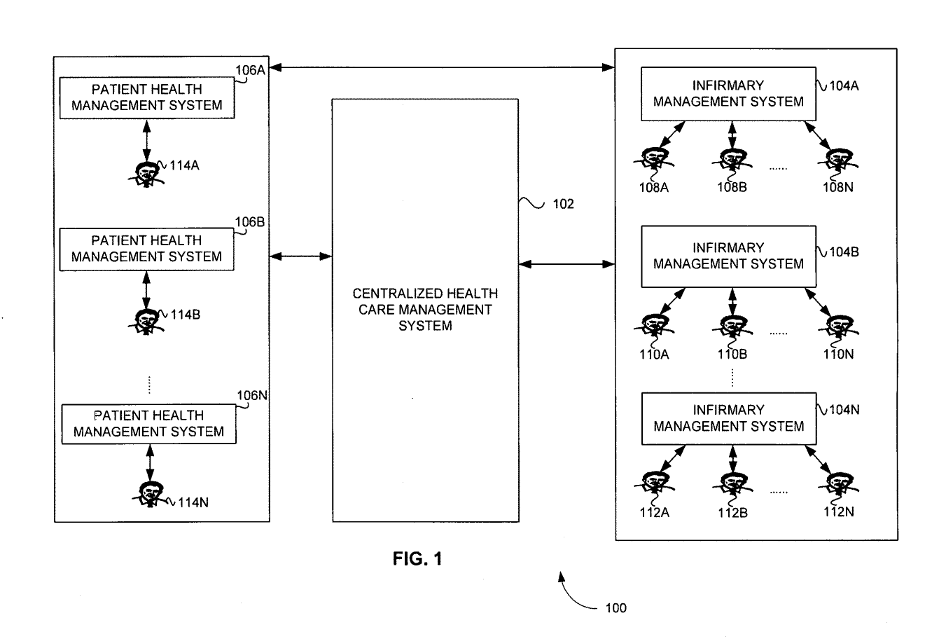

As mentioned, there remains a need for a system and method for managing a patient data. The embodiments herein achieve this by providing a patient data management system that provides options for physician's referrals, patient tagging, consolidation and standardization of healthcare data of one or more patients. Referring now to the drawings, and more particularly to FIGS. 1 through 20, where similar reference characters denote corresponding features consistently throughout the figures, there are shown preferred embodiments.

FIG. 1 illustrates a system view of a patient data management system 100 for managing data associated with one or more patients according to an embodiment herein. The patient data management system 100 includes a centralized healthcare management system 102, one or more infirmary management system 104A-N and one or more patient health management system 106A-N.

The one or more infirmary management system 104A-N includes one or more primary physicians 108A-N, and one or more secondary physicians 110A-N-112A-N. The one or more patient health management system 106A-N includes one or more patients 114A-N. The one or more patient health management system 106A-N may be designed to be a vendor agnostic digital health platform for multi-source health data integration and intelligence. The centralized healthcare management system 102 may be accessed by (i) the one or more primary physicians 108A-N, (ii) the one or more secondary physicians 110A-N and 112A-N and (iii) the one or more patients 114A-N. The centralized healthcare management system 102 may be a primary storage. The centralized healthcare management system 102 creates a single source of the data of the one or more patients 114A-N and act as a secondary storage for the data of the one or more patients 114A-N.

The centralized healthcare management system 102 enables and manages communication between the one or more infirmary management system 104A-N and the one or more patient health management system 106A-N. The one or more patient health management system 106A-N converts the data received from the one or more patients 114A-N into predefined standard data. The one or more patients 114A-N may download or view the patient data of the one or more patients 114A-N from the centralized healthcare management system 102. The centralized healthcare management system 102 collects the data of the one or more patients 114A-N from the one or more patient health management system 106A-N. In one embodiment, the one or more infirmary management system 104A-N collects the data of the one or more patients 114A-N from the one or more patient health management system 106A-N. The data may be a patient related data (e.g. health related information, heart beat rate, pulse rate, breath flow rate, blood related data, etc.)

The centralized healthcare management system 102 avoids possibility of a patient duplication by integrating the centralized healthcare management system 102 with Enterprise Master Patient Index (EMPI) and Identity Access Management (IAM). The one or more infirmary management system 104A-N allows the one more primary physicians 108A-N and the one or more patients 114A-N to communicate with each other. The one or more infirmary management system 104A-N shares the data of the one or more patients 14A-N with each other securely. The one or more infirmary management system 104A-N connects the one or more patients 114A-N with the centralized healthcare management system 102. The data of the one or more patients 114A-N may be automatically uploaded to the centralized healthcare management system 102 from the one or more patient health management system 106A-N.

The one or more patient health management system 106A-N updates the data of the one or more patients 114A-N into the centralized healthcare management system 102 in real time. The centralized healthcare management system 102 monitors the behaviour of the one or more patients 114A-N to collect the data from the one or more patients 114A-N based on the data received from the one or more patient health management system 106A-N in real time. The one or more patients 114A-N may control their data sharing with the one or more infirmary management system 104A-N.

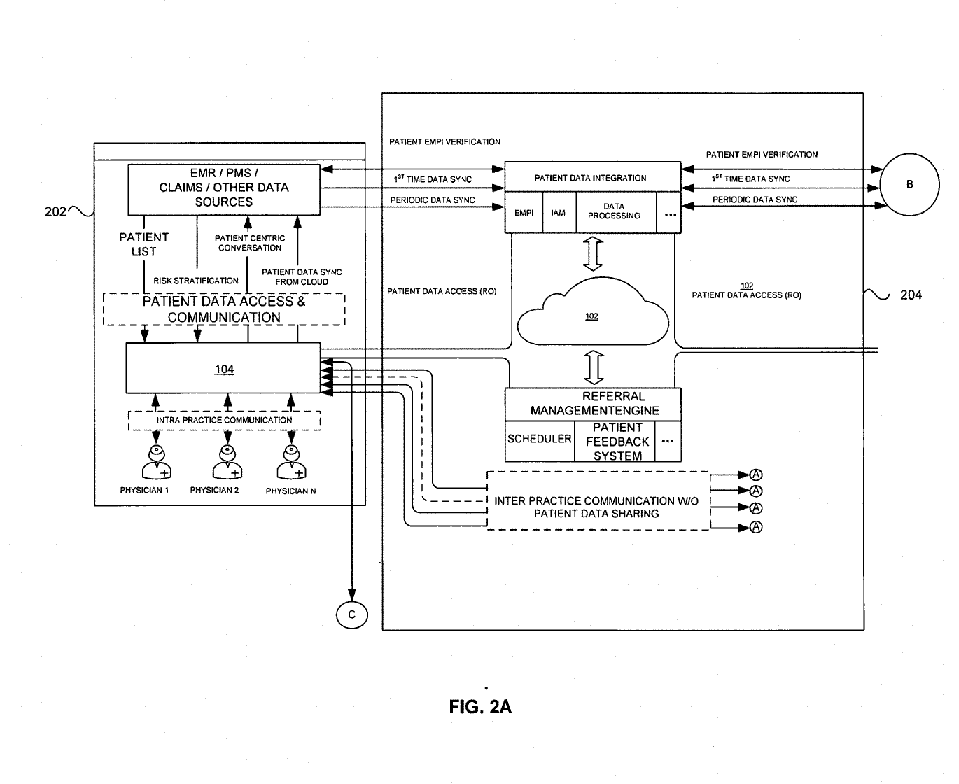

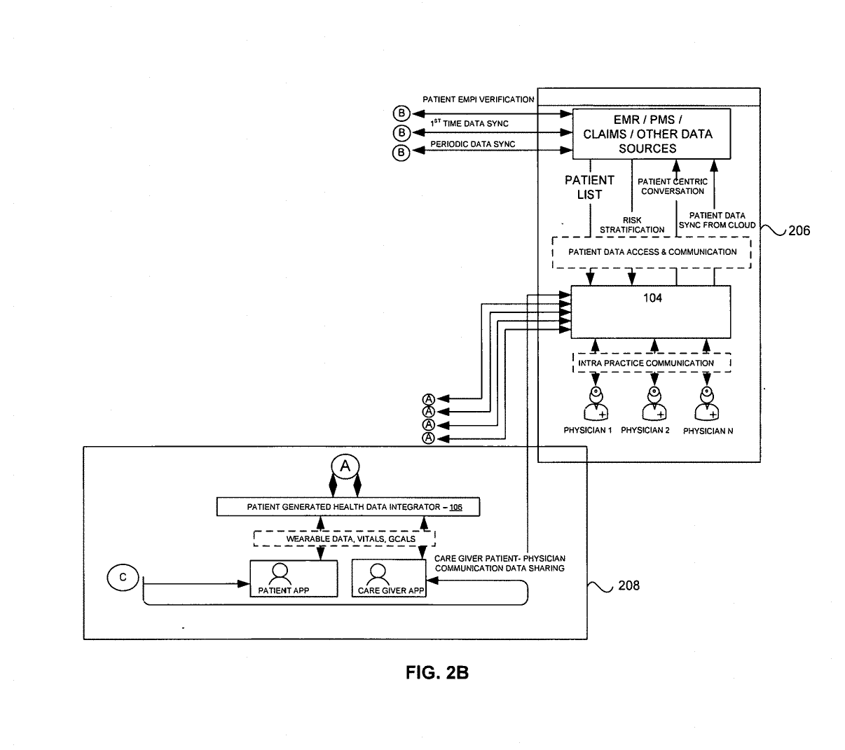

FIGS. 2A and 2B illustrate an architectural view of the patient data management system 100 of FIG. 1 according to an embodiment herein. The patient health management system 106A works for the patient 114A as a consumer as follows: (a) self-register or get invite from at least one physician and login to the patient health management system 106A in few simple steps; (b) connects all patients health devices (e.g. fitbit, viatom checkme, etc.) of the patient 114A and connect them with the patient 114A account under unique ID; (c) view or accept customized care plans created by the physician of the patient 114A; (d) obtains medications, goals, subjective data prescribed by the physician and updated within the patient health management system 106A once the care plan is accepted; (e) dynamically tracks the active devices of the one or more patients based on the data in the patient health management system 106A; (f) set own goals or let the physicians set the goals for the patient 114A based on the care plan; (g) dynamically monitors activities of the patient 114A by either auto update of the data from the multiple connected devices or manual entry of values in the patient health management system 106A; (h) enable the patient 114A to upload Continuity of Care Document (CCD) file or blue button file or Consolidated Clinical Document Architecture (CCDA) which the patients are eligible to get from any physician and maintain it themselves within their patient health management system 106A; (i) share the data and reports with the physician; (j) generate reports and share the reports with a care team and a circle of care. The patient health management system 106A is configured to (i) obtain a collective data from all patient active devices, (ii) record pain (e.g. cardiac arrest and muscle contraction) or any other symptoms, (iii) provide recommendation or answer to preventative goal maintenance questions which is pushed to the patient health management system 106A by the physician, and (iv) set/configure medication reminders.

The care team may be a clinical care team for a given patient consists of health professionals (e.g. physicians, advanced practice registered nurses, other registered nurses, physician assistants, clinical pharmacists, and other health care professionals) with the training and skills needed to provide high-quality, coordinated care specific to the patient's clinical needs and circumstances. The circle of care may be a term commonly used to describe the ability of certain health information custodians to assume an individual's implied consent to collect, use or disclose personal health information for the purpose of providing health care (in circumstances defined in PHIPA (The Personal Health Information Protection Act)); (l) the patient 114A or the consumer may also empower a caregiver or family member to perform one or more possible actions in the patient health management system 106A by sending them an invitation to download or view the patient health management system 106A and view, update or share data to provider on their behalf.

The one or more infirmary management system 104A-N transforms all data collected from multiple devices and sources into a standard format making the data easily actionable and accessible complying with the HIPAA (Health Insurance Portability and Accountability Act) guidelines. The one or more infirmary management system 104A-N reviews conversation occurred between the physicians about a patient (e.g. health tips for the patient, activities of the patent, vitals, etc) to help the primary physician to make more accurate diagnoses and engage more effectively with the patient in making treatment choices. The one or more infirmary management system 104A-N may assess an impact (e.g. results to the diagnosis) of wellness data (e.g. successful diagnosis to the diseases) on population health. The one or more infirmary management system 104A-N compares patient population treatment progress with the wellness data received from various medical systems and wearable devices for better disease management.

The one or more infirmary management system 104A-N improves clinical trials enhanced with behavioural data. For example, wearable devices for gathering human behavioural data and the adoption of connected medical devices as personal devices for vitals tracking is picking traction. However, the difficulty in integrating multiple vendor systems and devices and standardizing the data to make—easy accessible and actionable data. Hence clinical trial analysis, may be a best leverage this data which enable a more comprehensive view of the clinical trial participants in a seamless and hassle-free manner.

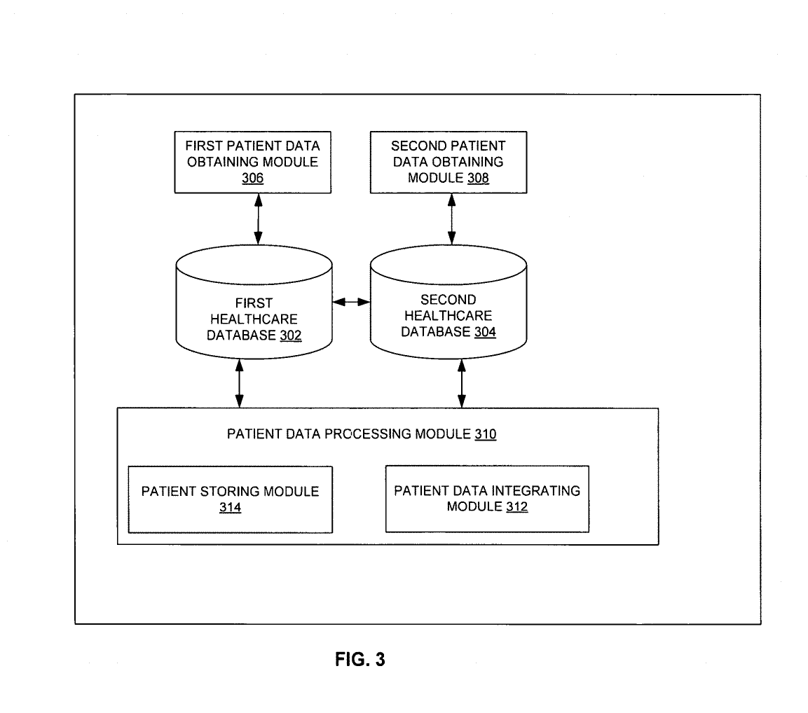

FIG. 3 illustrates an exploded view of the centralized healthcare management system 102 of the one or more patients 114A-N of FIG. 1 according to an embodiment herein. The exploded view of the centralized healthcare management system 102 includes a first healthcare database 302, a second healthcare database 304, a first patient data obtaining module 306, a second patient data obtaining module 308 and a patient data processing module 310. The first patient data obtaining module 306 obtains a first set of data from the one or more patient health management system 106A-N associated with the one or more patients 114A-N. The first set of data may be exposed by different wearable devices. The first set of data may be converted (i.e. in the centralized healthcare management system 102) as per required standard formats. The values of the first set of data may include source data (e.g., Fitbit and jawbone) based on the source data identifies the same and display it in the centralized healthcare management system 102.

The second patient data obtaining module 308 obtains a second set of data (e.g. patient health related information in infirmary standard) from the one or more infirmary management system 104A-N associated with one or more infirmary. The one or more infirmary management system 104A-N may communicate the second set of data to the one or more patient health management system 106A-N. The second set of data may be exposed by different wearable devices (e.g. Fitbit and jawbone). The second set of data may be converted as per required standard formats in the centralized healthcare management system 102.

The patient data processing module 310 processes the first set of data and the second set of data to consolidate or standardize (i) the first set of data and (ii) the second set of data. In one embodiment, the patient data processing module 310 consolidates or standardizes the first set of data and the second set of data to configure the single source data.

The data of the one or more patients 114A-N is standardized by the patient data management system 100 (e.g., if heart rate values come from the fitbit as just value, it will change to heart rate). The patient data processing module 310 checks units of the patient data and standardizes the unit of the patient data (e.g., for heart rate—bpm). The data processing module 310 may include a separate memory allocation for the data of the one or more patients 114A-N with the single source data including the unit and the data value. The data may be stored along with a patient unique ID.

The patient data processing module 310 includes a patient data integrating module 312 and a patient data storing module 314. The patient data integrating module 312 integrates the centralized healthcare management system 102 with the enterprise master patient index (EMPI) and the identity access management (JAM) to avoid duplication of the single source data of the one or more patients 114A-N. The centralized healthcare management system 102 includes a separate identity management server that handles a unique identification and access management part. For EMPI, the centralized healthcare management system 102 may include a duplication logic algorithm for identifying the data.

The patient data storing module 314 stores each of the first set of data and each of the second set of data in the first patient database 302 and the second patient database 304 separately. For example, when a patient is registered in the patient data management system 100, details of the patient may check in the IAM initially. In one embodiment, the patient data is stored separately in two databases (e.g. the first patient database 302, and the second patient database 304) to ensure maximum security to sensitive the patient information. The first healthcare database 302 stores all patient identifiable data including demographic and social information while patient's healthcare related information is stored in the second healthcare database 304.

Therefore, access to the first healthcare database 302 or the second patient database 304 is not constitute a breach or loss in the data. Authorized person (e.g. the patients or the providers) may access the data by using an identification key unique to each patient. If the patient exists, checking database of the EMPI and mark the different sources (for e.g. EMR1, EMR 2) against the patient are needed instead of creating another new duplicate record. For the new patient, the new patient has to create a new record with UUID (Universal Unique Identifier).

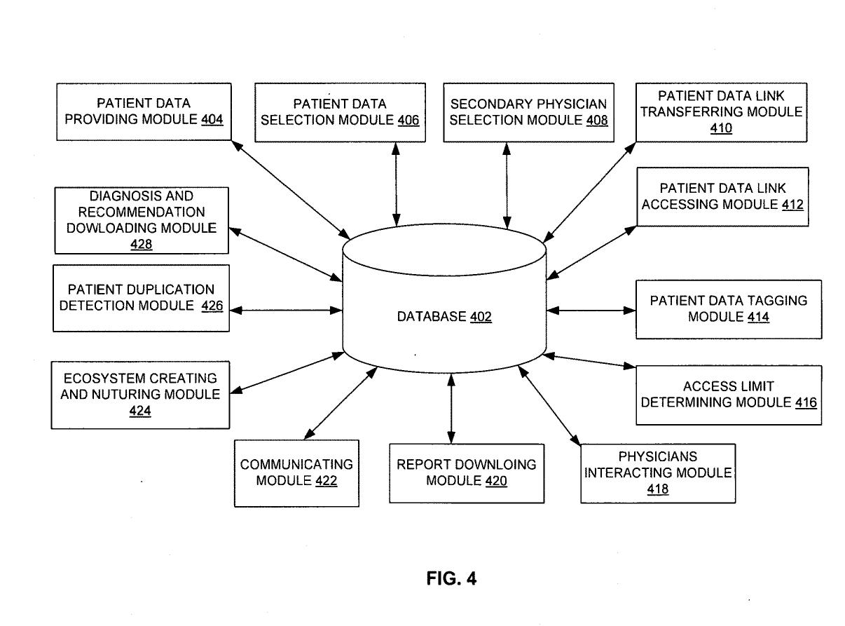

FIG. 4 illustrates an exploded view of the one or more infirmary management system 104A-N of FIG. 1 according to an embodiment herein. The exploded view of the one or more infirmary management system 104A-N includes a database 402, a patient data providing module 404, a patient data selection module 406; a secondary physician selection module 408, a patient data link transferring module 410, a patient data link accessing module 412, a patient data tagging module 414, an access limit determining module 416, physicians interacting module 418, a report downloading module 420, a communicating module 422, an ecosystem creating and nurturing module 424, a patient duplication detection module 426, and a diagnosis and recommendation downloading module 428.

The patient data providing module 404 provides a list of the data. The patient data selection module 406 selects at least one data from the list of the data to view the data of the at least one the data. The patient medical history includes at least one of a medical treatment data related to the patient, conversation or discussion about the data with one or more secondary physicians 110A-N and 112A-N. The secondary physician selection module 408 selects the one or more secondary physicians 110A-N and 112A-N to initiate a conversation with respect to the first set of data and/or the second set of data. The patient data link transferring module 410 transfers a link associated with the first set of data and/or the second set of data to selected the one or more secondary physicians 110A-N and 112A-N.

For example, the data may be shared as the link with the one or more secondary physicians 110A-N and 112A-N via chat (e.g. communication). When the one or more secondary physicians 110A-N and 112A-N clicks on the link, the patient data management system 100 notifies the one or more primary 108A-N stating the one or more secondary physicians 110A-N and 112A-N (e.g., provider B) is requesting for access (approve/reject). For approval, the one or more secondary physicians 110A-N and 112A-N able to access the one or more patients detail. The patient data link accessing module 412 enables access the link by requesting to the primary physician.

For example, when there is a transfer of the data from one physician to the other physician, unique link may be generated. The link may be a combination of both physician ids. The one or more secondary physicians 110A-N and 112A-N may click on the link and access the data or may also request for access (if he has no access to patient details).

The one or more primary physicians 108A-N (e.g., provider A) may approve the request after when the one or more secondary physicians 110A-N and 112A-N) view the details. The patient data tagging module 414 tags the first set of data and/or the second set of data of the one or more patients 114A-N with selected each of the one or more secondary physicians 110A-N and 112A-N under unique id. The access limit determining module 416 determines a limit of access of the one or more data segments of the first set of data and/or the second set of data of the one or more patients 114A-N with respect to the one or more secondary physicians 110A-N and 112A-N by selecting each of the one or more secondary physicians 110A-N and 112A-N separately based on their field of expertise. The physicians interacting module 418 interacts with the one or more secondary physicians 110A-N and 112A-N about the first set of data and/or the second set of data of the one or more patients.

The report downloading module 42Q allows the one or more secondary physicians 110A-N and 112A-N to download or view or edit an electronic medical report associated with the first set of data and/or the second set of data. The communicating module 422 allows the one or more patients to communicate with each other. The first set of data and/or the second set of data may be transferred using a unique link. The ecosystem creating and nurturing module 424 creates and nurtures an ecosystem of the one or more physicians who work together. The purpose of including such the ecosystem, consolidating the first set of data and/or the second set of data from different EMRs or in other words single source of entity will enable patients to get complete health history whenever required. For physicians (e.g. providers) they may also get complete health history of patient so that appropriate consultation to avoid duplication which will in-turn be cost effective.

The patient duplication detecting module 426 detects the data associated with the one or more patients to avoid patient duplication by integrating with the EMPI and the IAM. The diagnosis and recommendation downloading module 428 uploads the diagnosis and recommendation are given by the one or more secondary physicians to the one or more infirmary management system 104A-N dynamically or periodically. Diagnosis and any patient related data may upload from EMR when the integration is processed. The uploading process performs through a Health Level-7 or Health Level version 7 or HL7 interface ((Application Programming Interface (API Integration)) done between the centralized healthcare management system 102 and the respective EMR system. The HL7 refers to a set of international standards for transfer of clinical and administrative data between software applications used by various healthcare providers. These standards focus on the application layer, which is “layer 7” in the Open Systems Interconnection (OSI) model. The API refers to a set of subroutine definitions, protocols, and tools for building software application.

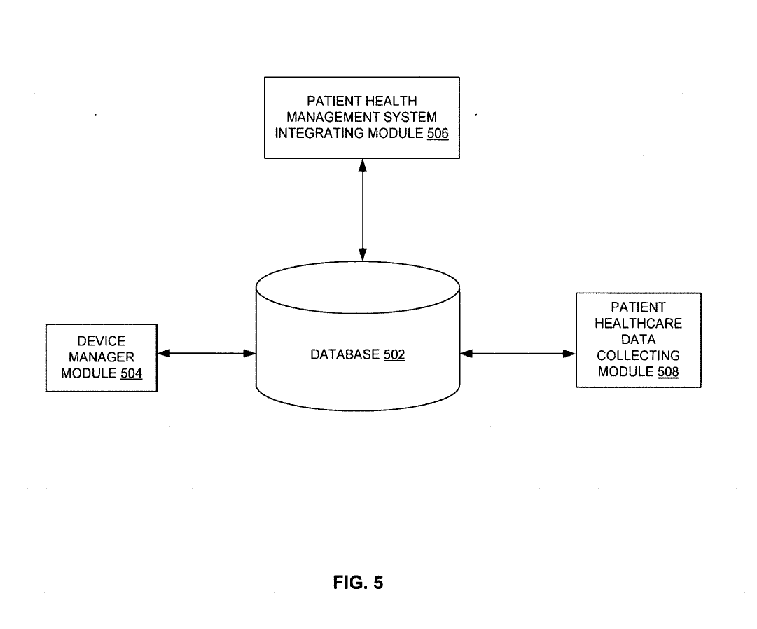

FIG. 5 illustrates an exploded view of one or more patient health management system 106A-N of FIG. 1 according to an embodiment herein. The exploded view of the one or more patient health management system 106A-N includes a database 502, a device manager module 504, a patient health management system integrating module 506 and a patient data collecting module 508. The device manager module 504 enables a list of all available devices which are supported to the one or more patient health management system 106A-N. The primary provider may authorize (e.g. via secure link) the secondary provider to access the patient data. The patient health management system integrating module 506 integrates the one or more patient health management system 106A-N with one or more healthcare monitoring devices to obtain the data and to communicate the first set of data and/or the second set of data to their the one or more of primary physicians. The patient data collecting module 508 collects the data in non-invasive manner to track the first set of data and/or the second set of data by the one or more physicians.

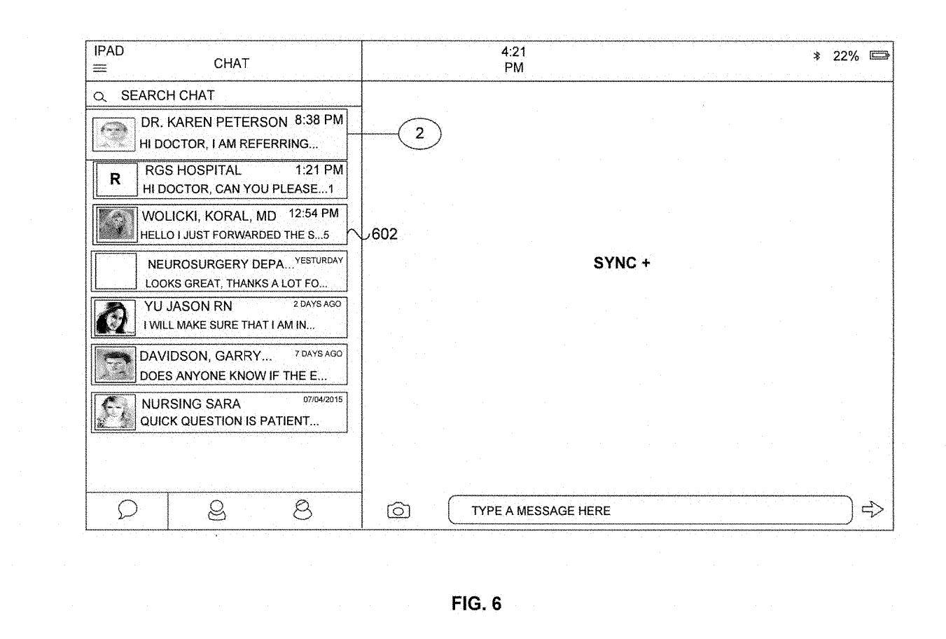

FIG. 6 illustrates a user interface view of a patient selection of the one or more infirmary management system 104A-N according to an embodiment herein. The user interface view of the patient selection includes a patient list 602. The patient list may include an information associated with the one or more patients. The primary physicians 108A-N may select a patient from the patient list 602 to initiate a secure communication with other physicians.

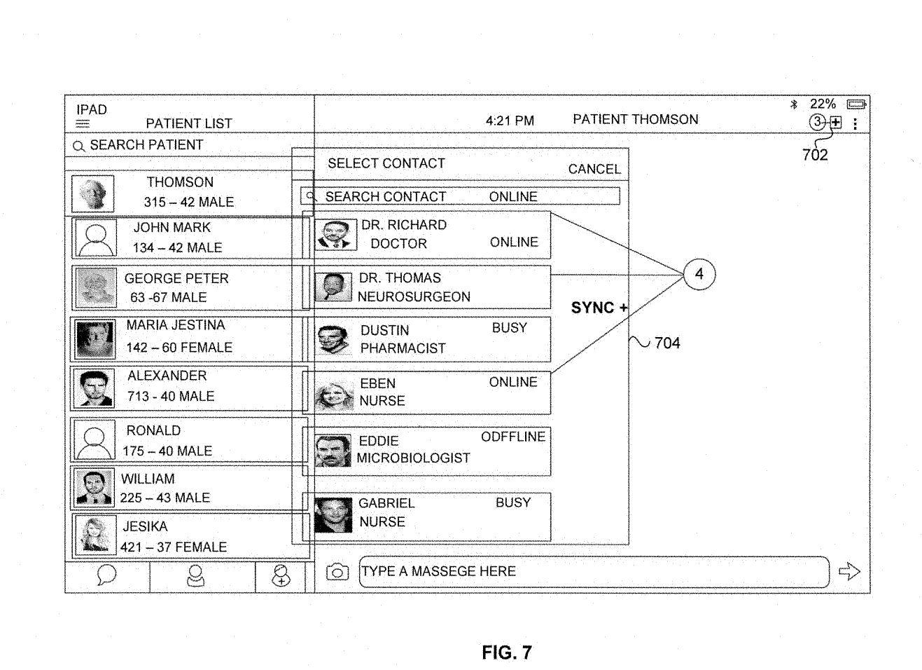

FIG. 7 illustrates a user interface view of a tag physicians of the one or more infirmary management system 104A-N according to an embodiment herein. The user interface view includes a healthcare professionals list 702 and a provider search tab 704. The one or more primary physicians 108A-N can see the healthcare professionals list by clicking the healthcare professionals list tab 702. The healthcare professionals list tab 702 may locate on the top right corner on the tag physicians. The provider search tab 704 includes one or more secondary physicians 110A-N and 112A-N. One or more primary physicians 108A-N may search for the one or more secondary physicians 110A-N and 112A-N or select one or more secondary physicians 110A-N and 112A-N relevant to the case to initiate parallel chats (e.g. messaging or audio or video communication). The one or more primary physicians 108A-N may search available physicians (e.g. online, busy) in the provider search tab 704.

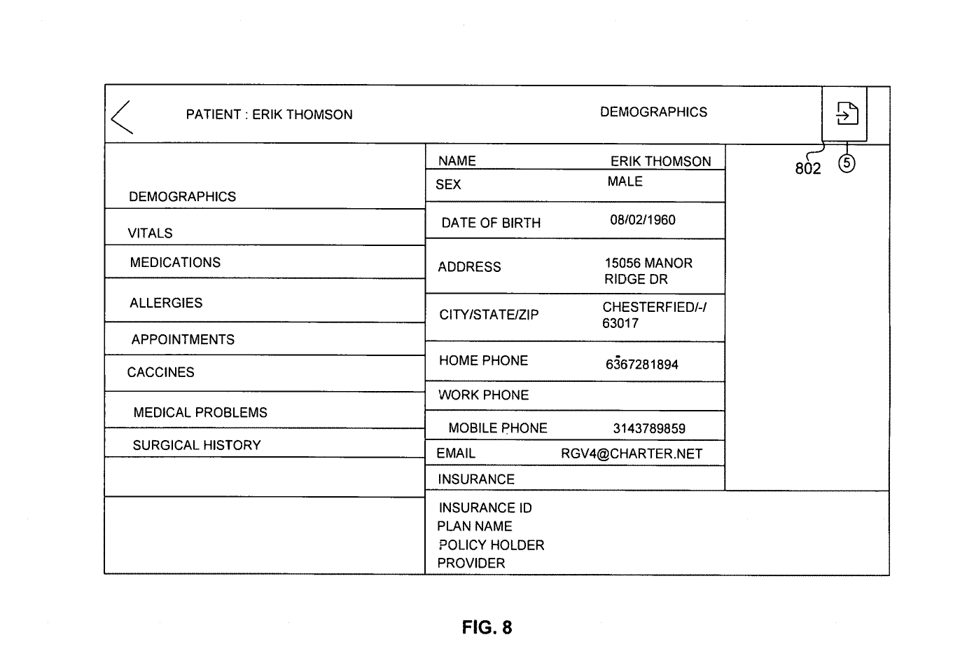

FIG. 8 illustrates a user interface view of a data sharing according to an embodiment herein. The user interface view of a data sharing includes a patient data sharing tab 802. The one or more primary physicians 108A-N may share the patient data with another provider by clicking the patient data sharing tab 802 with the one or more secondary physicians 110A-N and 112A-N. The patient data sharing tab 802 may be located on top right corner of the patient data sharing.

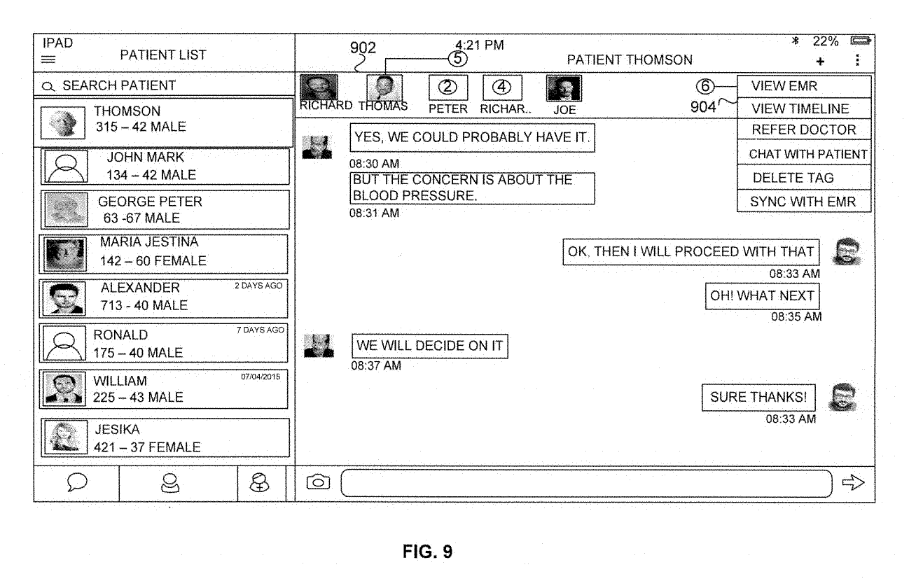

FIG. 9 illustrates a user interface view of a selectively dispense data according to an embodiment herein. The user interface yiew of a selectively dispense data includes a privy communication tab 902 and a view EMR tab 904. The one or more primary physicians 108A-N selects the one or more secondary physicians 110A-N and 112A-N by selecting each one or more secondary providers separately, and sharing the data, which is relevant to the field (e.g. Cardiologist, Dental, etc.) of the secondary physicians 110A-N and 112A-N. The view EMR tab 904 allows the primary provider to access the data. The primary physician may assign the link to a particular patient will have access to the data. The privy communication tab 902 enables the one or more primary physicians 108A-N selects the one or more secondary physicians 110A-N to communicate with each other about the data securely in real time.

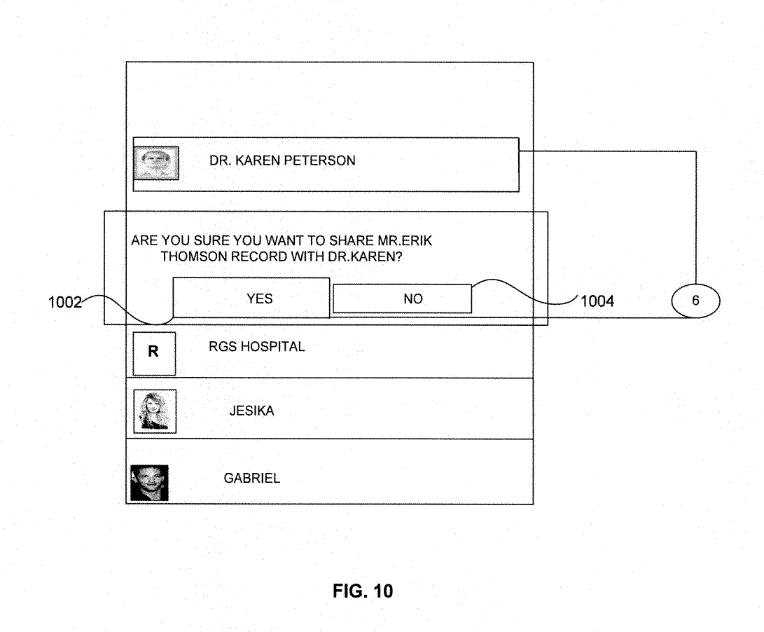

FIG. 10 illustrates a user interface view of a patient data transfer confirmation according to an embodiment herein. The user interface view of the data transfer confirmation includes a YES tab 1002 and NO tab 1004. The YES tab 1002 approves the data transfer, if the YES tab 1002 is selected and the NO tab 1004 denies the data transfer, if the NO tab 1004 is selected.

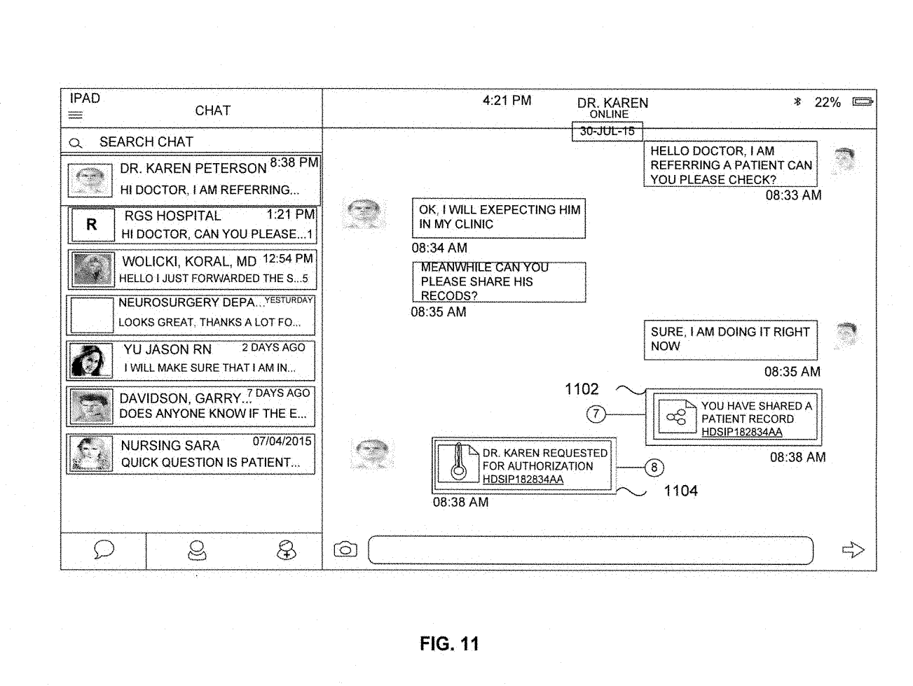

FIG. 11 illustrates a user interface view of a patient data link sharing according to an embodiment herein. The user interface view of the patient healthcare data link includes a patient data link tab 1102 and a request for authorization tab 1104. The patient data link tab 1102 share the data via link securely. The secondary physician may send a request to the primary physician via authorization tab 1104. The secondary physician may view or download the data once the request is approved by the primary physician.

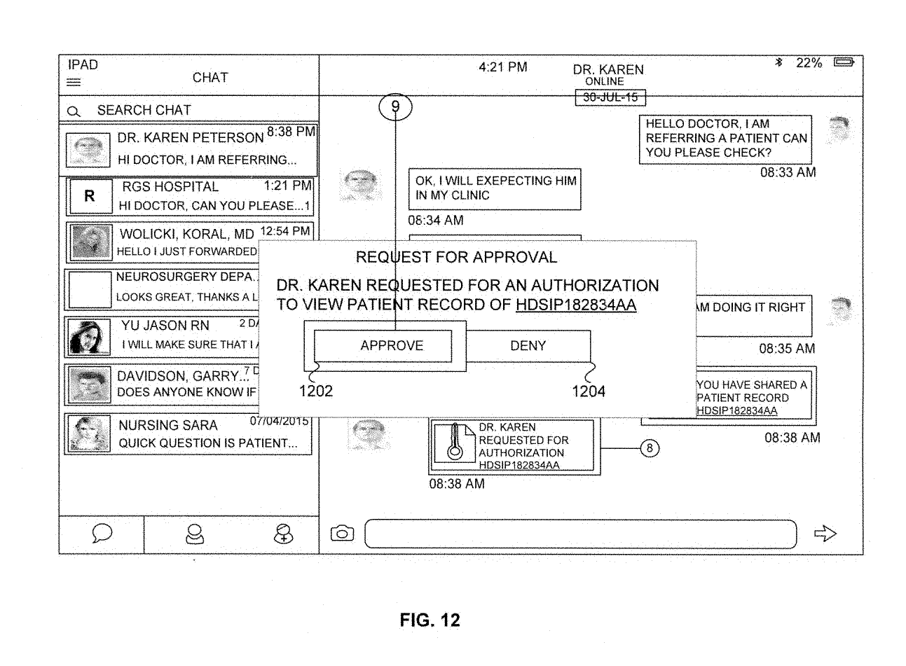

FIG. 12 illustrates a user interface view of a request for approval according to an embodiment herein. The user interface view of the request for approval includes an approve tab 1202 and a deny tab 1204. The one or more primary physicians 108A-N receives a request from the one or more secondary physicians 110A-N and 112A-N to access the data. The one or more primary physicians 108A-N may approve the request of the one or more secondary physicians 110A-N and 112A-N by clicking the approve tab 1202 in the pop-up menu. In one embodiment, the one or more primary physicians 108A-N may deny the request of the one or more secondary physicians 110A-N and 112A-N by clicking the deny tab 1204 in the pop-up menu.

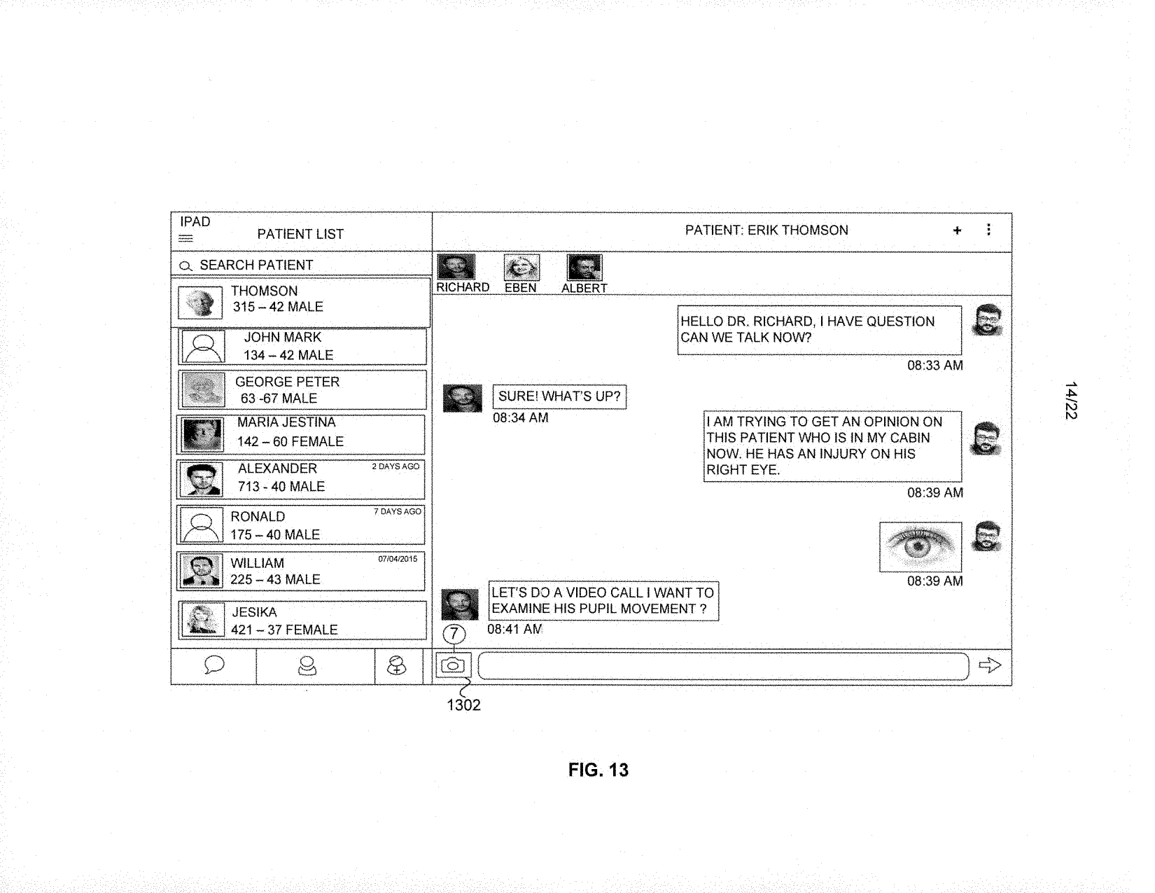

FIG. 13 illustrates a user interface view of a patient data sharing according to an embodiment herein. The user interface view of the data sharing includes a camera tab 1302. The one or more primary physicians 108A-N may share the specific data (e.g., images, videos and reports) using camera tab 1302.

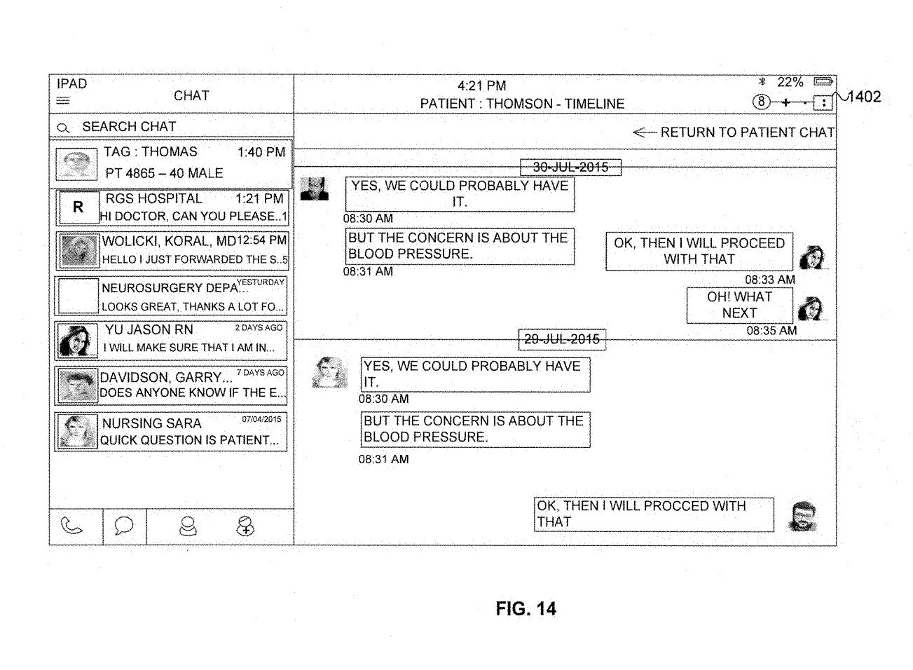

FIG. 14 illustrates a user interface view of a history of the patient according to an embodiment herein. The user interface view includes a conversation history view tab 1402. The primary physician may view past conversation about the patient using the conversation history view tab 1402. The conversation history view tab 1402 may locate on the top and right corner of the user interface view of the timeline tab 1400. The conversation history view tab 1402 displays conversation history between the one or more primary physicians 108A-N and the one or more secondary physicians 110A-N and 112A-N.

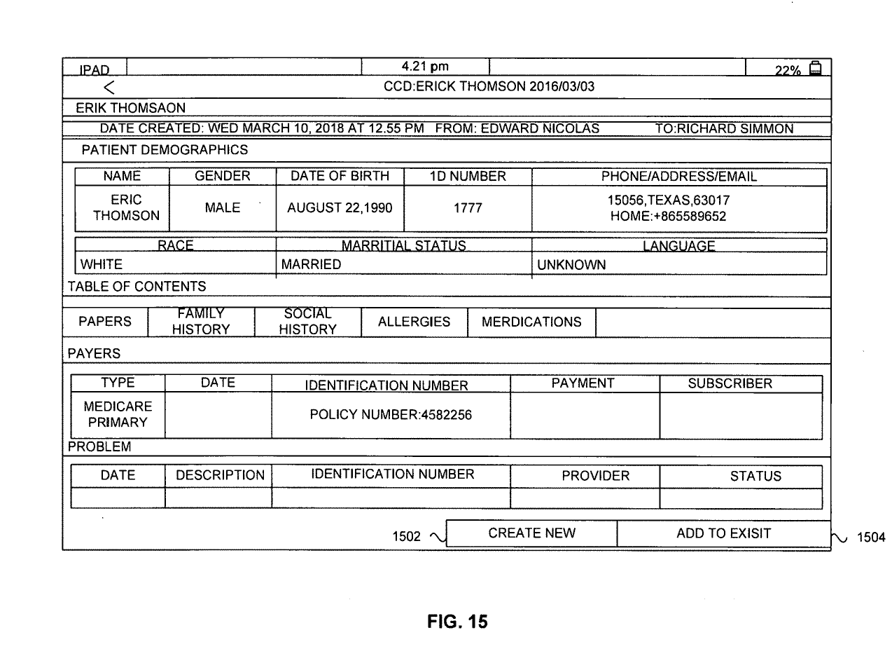

FIG. 15 illustrates a user interface view of an electronic health record/electronic medical record (EHR/EMR) view with one or more action according to an embodiment herein. The user interface view of the EHR/EMR view includes a create new tab 1502 and an existing tab 104. Once approved by the one or more primary physicians 108A-N, the one or more secondary physicians 110A-N and 112A-N may access a EHR/EMR data. The one or more secondary physicians 110A-N and 112A-N may save the EHR/EMR data for future purpose by clicking the create new tab 1502 and add to the existing tab 1504. The create new tab 1502 enables the user to create new EHR/EMR. The create new tab 1502 the existing tab 1504 may locate in the right down of the EHR/EMR view.

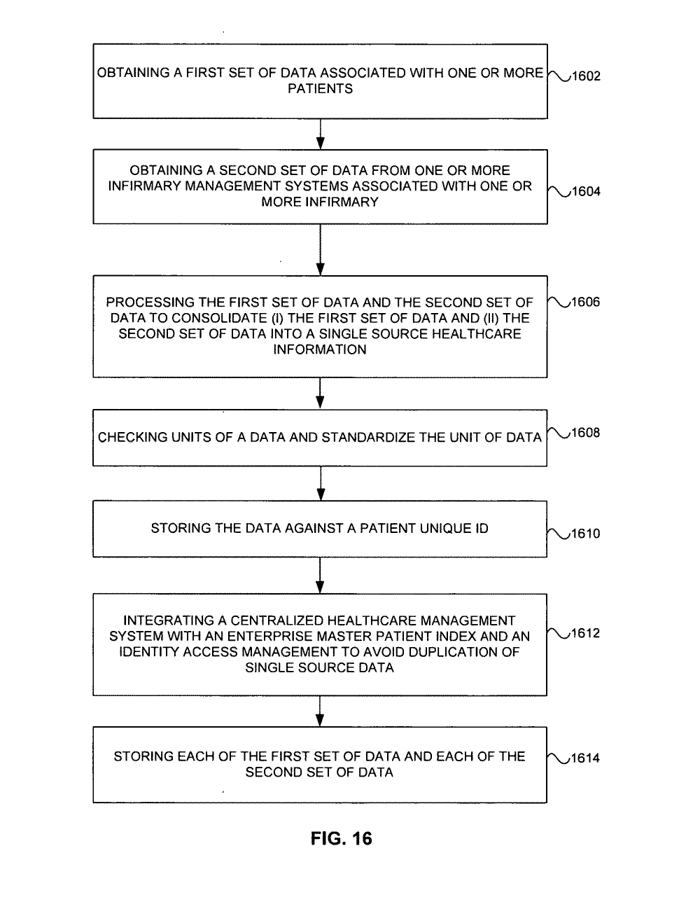

FIG. 16 is a flow diagram illustrating a method of managing healthcare data using the centralized healthcare management system 102 of FIG. 1 according to an embodiment herein. At step 1602, a first set of data is obtained. At step 1604, a second set of data is obtained from the one or more infirmary management systems 104A-N associated with one or more infirmary. At step 1606, the first set of data and the second set of data are processed to consolidate (i) the first set of data and (ii) the second set of data into a single source data. At step 1608, units of data are checked and the unit of data is standardized. At step 1610, the data is stored against a patient ID. At step 1612, the centralized healthcare management system 102 is integrated with an enterprise master patient index and an identity access management to avoid duplication of single source data. At step 1614, each of the first set of data and each of the second set of data is stored.

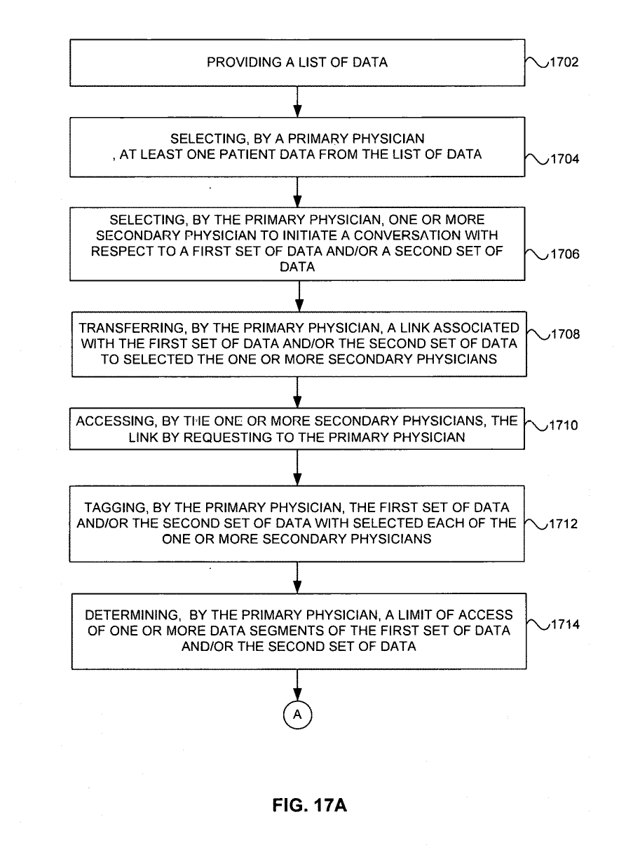

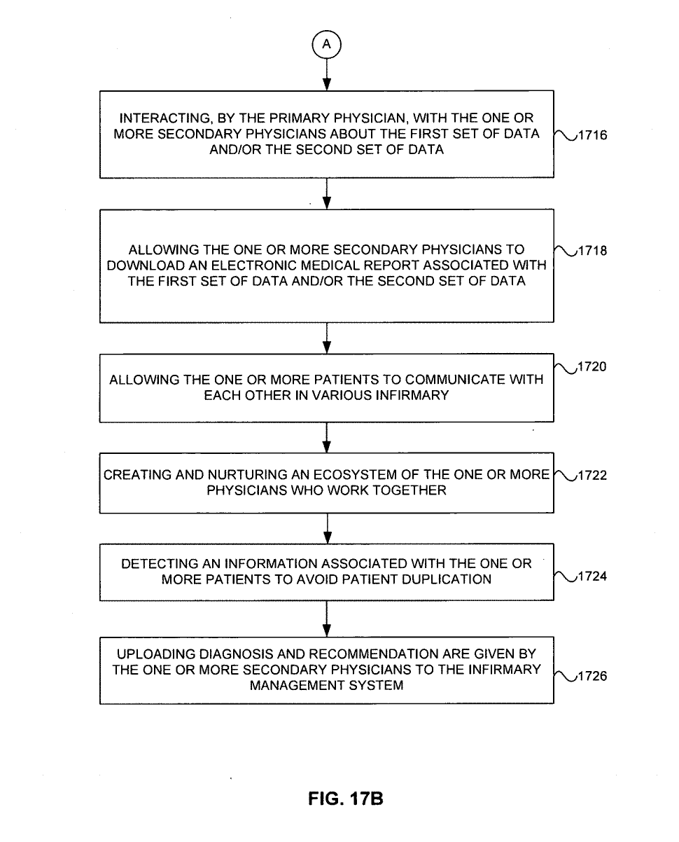

FIGS. 17A and 17B are flow diagrams illustrating a method of managing healthcare data using the one or more infirmary management system 104A-N of FIG. 1 according to an embodiment herein. At step 1702, a list of data is provided by the one or more infirmary system 104A-N. At step 1704, at least one data is selected from the list of data by the primary physician to view a patient medical data of the at least one data. At step 1706, one or more secondary physicians 110A-N and 112A-N is selected by the primary physician 108A-N to initiate a conversation with respect to a first set of data and/or a second set of data. At step 1708, a link associated with the first set of data and/or the second set of data is transferred by the primary physician to selected the one or more secondary physicians 110A-N and 112A-N.

At step 1710, the link by requesting to the primary physician 108A-N is accessed by the one or more secondary physicians 110A-N and 112A-N. At step 1712, the first set of data and/or the second set of data is tagged with selected each of the one or more secondary physicians 110A-N and 112A-N by the primary physician 108A-N. At step 1714, a limit of access of one or more data segments of the first set of data and/or the second set of data is determined by the primary physician.

At step 1716, the one or more secondary physicians 110A-N and 112A-N are interacted with the primary physician 108A-N about the first set of data and/or the second set of data. At step 1718, the one or more secondary physicians 110A-N and 112A-N downloads an electronic medical report associated with the first set of data and/or the second set of data. At step 1720, the one or more patients 114A-N allowed to communicate with each other in various infirmary. At step 1722, an ecosystem of the one or more physicians is created and nurtured who work together. At step 1724, a data is detected associated with the one or more patients to avoid patient duplication. At step 1726, diagnosis and recommendation are given by the one or more secondary physicians 110A-N and 112A-N are uploaded to the one or more infirmary management system 104A-N.

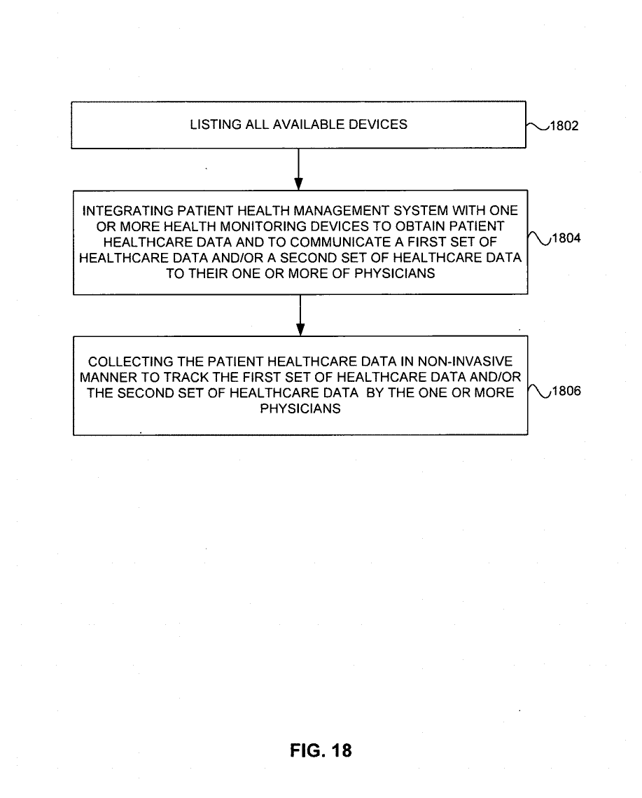

FIG. 18 is a flow diagram illustrating a method of managing healthcare data using the one or more patient health management system 106A-N of FIG. 1 according to an embodiment herein. At step 1802, all available devices are listed. At step 1804, the patient health management system 106A-N with one or more healthcare monitoring devices are integrated to obtain the data and to communicate the first set of data and/or the second set of data to their the one or more of physicians. At step 1806, the data in non-invasive manner is collected to track the first set of data and/or the second set of data by the one or more physicians.

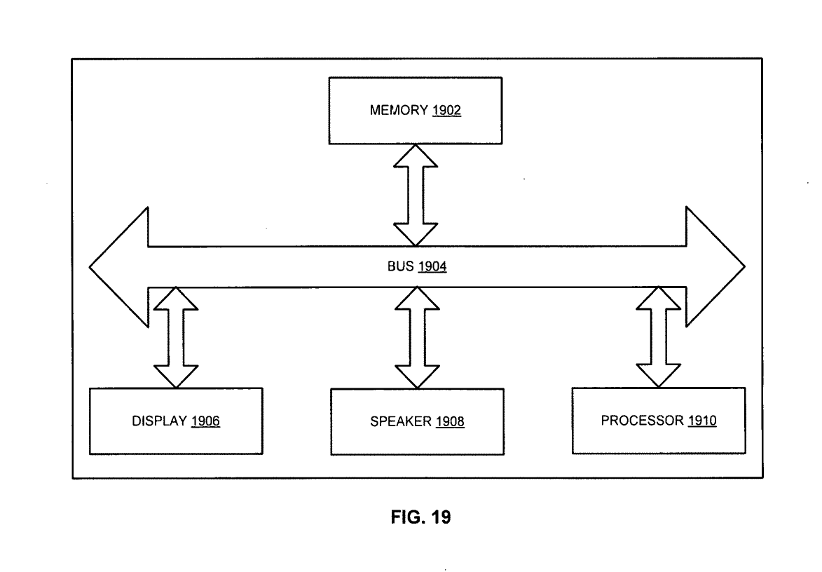

FIG. 19 illustrates an exploded view of a receiver 1900 of FIG. 1 having a memory 1902 having a set of instructions, a bus 1904, a display 1906, a speaker 1908 and a processor 1910 capable of processing the set of instructions to perform any one or more of the methodologies herein, according to an embodiment herein. The processor 1910 may also enable digital content to be consumed in the form of video for output via one or more displays 1906 or audio for output via speaker and/or earphones 1908. The processor 1910 may also carry out the methods described herein and in accordance with the embodiments herein.

Digital content may also be stored in the memory 1902 for future processing or consumption. The memory 1902 may also store program specific information and/or service information (PSI/SI), including information about digital content (e.g., the detected information bits) available in the future or stored from the past. A user of the receiver 1900 may view this stored information on display 1906 and select an item of for viewing, listening, or other uses via input, which may take the form of keypad, scroll, or other input device(s) or combinations thereof. When digital content is selected, the processor 1910 may pass information. The content and PSI/SI may be passed among functions within the receiver using the bus 1904.

The techniques provided by the embodiments herein may be implemented on an integrated circuit chip (not shown). The chip design is created in a graphical computer programming language, and stored in a computer storage medium (such as a disk, tape, physical hard drive, or virtual hard drive such as in a storage access network). If the designer does not fabricate chips or the photolithographic masks used to fabricate chips, the designer transmits the resulting design by physical means (e.g., by providing a copy of the storage medium storing the design) or electronically (e.g., through the Internet) to such entities, directly or indirectly.

The stored design is then converted into the appropriate format (e.g., GDSII) for the fabrication of photolithographic masks, which typically include multiple copies of the chip design in question that are to be formed on a wafer. The photolithographic masks are utilized to define areas of the wafer (and/or the layers thereon) to be etched or otherwise processed.

The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher-level carrier) or in a multichip package (such as a ceramic carrier that has either or both surface interconnections or buried interconnections). In any case the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

The embodiments herein can take the form of, an entirely hardware embodiment, an entirely software embodiment or an embodiment including both hardware and software elements. The embodiments that are implemented in software include but are not limited to, firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (I/O) devices (including but not limited to keyboards, displays, pointing devices, remote controls, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

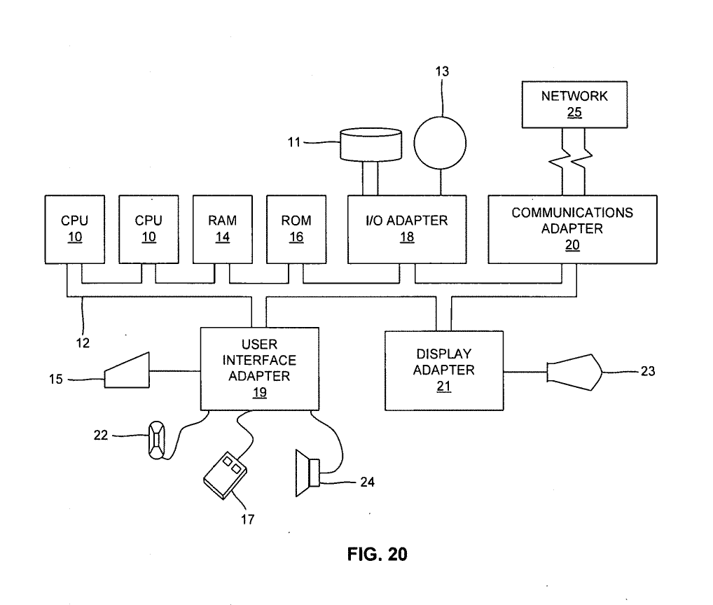

A representative hardware environment for practicing the embodiments herein is depicted in FIG. 20. This schematic drawing illustrates a hardware configuration of an information handling/computer system in accordance with the embodiments herein. The system comprises at least one processor or central processing unit (CPU) 10. The CPUs 10 are interconnected via system bus 12 to various devices such as a random-access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system can read the inventive instructions on the program storage devices and follow these instructions to execute the methodology of the embodiments herein.

The system further includes a user interface adapter 19 that connects a keyboard 15, mouse 17, speaker 24, microphone 22, and/or other user interface devices such as a touch screen device (not shown) or a remote control to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

The advantages of the data management system 100 as follows: (a) care plan management solution. A customized care plan is shared for the patient by the physician and an ability to continuously monitor its adherence, once the patient accepts the care plan and enters data manually or using wearable devices; (b) patient centric conversation (e.g. patient tagging). Ability for one Physician to communicate (e.g. chat) with other physician about a patient by tagging their name to the chat thereby managing a history of conversations done against a patient for maintaining continuity of care; (c) referral closure loop with multiple tracking status-ability to get referral list from integrated EMR's or manually initiate a referral and track the referral status with the referred physician till closure. Also Sync the consultation notes or feedback to referred physician's EMR if required; (d) capability of data consolidation and standardization—data integration from multiple sources and presenting the data in a standardized format; (e) suggestion on appropriate physician based on intelligent search algorithm. During referrals physicians may be search based on various parameters such as Location, NPI, Insurance, etc., In addition to that when a patient is selected, the search algorithm of the system displays the list of all physicians associated with that patient for last six months fetching the data from EMR. This enables referring physician to get the history of specialists from whom patient has got care from and also paves the way for referring the patient again to the specialist who is aware of patient clinical history.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope.

I/We claim:

1. A computer implemented method for secure communication of healthcare data related to a patient by a primary physician with one or more secondary physicians using a secure healthcare data management server (SHDMS), wherein the method comprises:
selecting, by the primary physician, a first patient information among a list of patients, wherein each physician acts as a primary physician for the list of patients;
enabling the primary physician to tag the first patient information with one or more secondary physicians based on the field of expertise of each secondary physician;
enabling the primary physician to initiate a communication with the tagged one or more secondary physicians for diagnosis or request help in treatment by sharing electronic medical report (EMR) of the patient;
determining a field of expertise of the tagged one or one or more secondary physicians;
enabling the primary physician to limit access to the EMR of the patient to the tagged one or more secondary physicians by selecting each physician separately that is specifically relevant to the field of expertise of the tagged one or one or more secondary physicians;
identifying, by at least one tagged secondary physician, a disease or ailment of the first patient based on the EMR from the primary physician; and
recommending, by the least one tagged secondary physician, a customized treatment process for the first patient based on the identified disease of the first patient to the primary physician.

2. The method as claimed in claim 3, further comprising:
enabling the primary physician to view a history of communication in a timeline window associated with the one or more secondary physicians associated with each patient in a predetermined time manner.

3. The method as claimed in claim 1, further comprising:
verifying the one or more secondary physicians by communicating the patient data as a secured link from the primary physician to the one or more secondary physicians;
authorizing the secure link by the one or more secondary physicians to access the patient data; and
enabling one or more secondary physicians to access EMR through SHDMS when the secure link is accessed by the one or more secondary physicians.

4. The method as claimed in claim 1, further comprising:
creating communication groups from tagged one or more secondary physicians based on their field of expertise and the patient data.

5. The method as claimed in claim 4, further comprising:
enabling the primary physician to select the one or more secondary physicians in at least one communication group; and
enabling at least one selected secondary physician to view and access the patient data in the at least one communication group even if the one or more secondary physicians are present in the group by sharing a secure link to the selected secondary physician.

6. A secure healthcare data management server (SHDMS) for secure communication of healthcare data related to a patient by a primary physician with one or more secondary physicians, wherein the server comprises:
a memory unit that stores a database and a set of modules; and
a processor that executes set of modules, wherein set of modules comprise:
a patient data selection module, executed by the processor, configured to select a first patient information among a list of patients, by the primary physician, wherein each physician acts as a primary physician for the list of patients;
a secondary physician selection module, executed by the processor, configured to enable the primary physician to tag the first patient information with one or more secondary physicians based on the field of expertise of each secondary physician;
a physician interacting module, executed by the processor, configured to enable the primary physician to initiate a communication with the tagged one or more secondary physicians for diagnosis or request help in treatment by sharing electronic medical report (EMR) of the patient;
a patient data tagging module, executed by the processor, configured to determine a field of expertise of the tagged one or one or more secondary physicians;
an access limit determining module, executed by the processor, configured to enable the primary physician to limit access to the EMR of the patient to the tagged one or more secondary physicians by selecting each physician separately that is specifically relevant to the field of expertise of the tagged one or one or more secondary physicians; and
a diagnosis and recommendation downloading module, executed by the processor, configured to identify a disease or ailment of the first patient based on the EMR from the primary physician, by at least one tagged secondary physician, wherein the diagnosis and recommendation downloading module recommending a customized treatment process for the first patient based on the identified disease of the first patient to the primary physician by the least one tagged secondary physician.

7. The server as claimed in claim 6, further the physician interacting module executed by the processor, configured to enable the primary physician to view a history of communication in a timeline window associated with the one or more secondary physicians associated with each patient in a predetermined time manner.

8. The server as claimed in claim 6, further the physicians interacting module executed by the processor, configured to
verify the one or more secondary physicians by communicating the patient data as a secured link from the primary physician to the one or more secondary physicians;
authorize the secure link by the one or more secondary physicians to access the patient data; and
enable one or more secondary physicians to access EMR through SHDMS when the secure link is accessed by the one or more secondary physicians.

9. The server as claimed in claim 6, further the access limit determining module, executed by the processor, configured to create communication groups from tagged one or more secondary physicians based on their field of expertise and the patient data.

10. The server as claimed in claim 6, further the secondary physician selection module, executed by the processor configured to enable the primary physician to select the one or more secondary physicians in at least one communication group.

11. The server as claimed in claim 6, further a patient data link accessing module, executed by the processor configured to enable at least one selected secondary physician to view and access the patient data in the at least one communication group even if the one or more secondary physicians are present in the group by sharing a secure link to the selected secondary physician.
